(19) United States
(12) Patent Application Publication
(10) Pub. No.: US 2014/0234430 A1
(43) Pub. Date: Aug. 21, 2014

(54) PHARMACEUTICAL METHODS AND TOPICAL COMPOSITIONS CONTAINING ACITRETIN

(71) Applicant: DOUGLAS PHARMACEUTICALS LTD., Auckland (NZ)

(72) Inventors: Peter Surman, Henderson (NZ); Fergus Cameron Binnie, Henderson (NZ); Marten Geert Vos, Glen Eden (NZ)

(21) Appl. No.: 14/347,744

(22) PCT Filed: Oct. 1, 2012
(86) PCT No.: PCT/IB12/02454

§ 371 Date: Mar. 27, 2014

Related U.S. Application Data
(60) Provisional application No. 61/543,484, filed on Oct. 5, 2011.

(51) Int. Cl. A61K 31/202

(52) U.S. Cl. CPC A61K 31/202
USPC 424/490; 562/471; 514/570

(57) ABSTRACT

The present invention is directed to methods and compositions for topical administration of acitretin. More specifically, the present invention is related to methods and compositions for the treatment or prevention or reduction of symptoms or signs of dermatological conditions using acitretin in a topical administration. More specifically, the present invention is related to methods and compositions containing acitretin which are effective for the treatment or prevention or reduction of symptoms or signs of keratoses, in particular actinic keratosis.

### BACKGROUND

1. Field

The present invention is directed to methods and compositions for topical administration of acitretin. More specifically, the present invention is related to methods and compositions for the treatment or prevention or reduction of symptoms or signs of dermatological conditions using acitretin in a topical administration. More specifically, the present invention is related to methods and compositions containing acitretin which are effective for the treatment or prevention or reduction of symptoms or signs of keratoses, in particular actinic keratosis.

2. Description of the Related Art

a. Acitretin

Acitretin ((2E,4E,6E,8E)-9-(4-methoxy-2,3,6-trimethylphenyl)-3,7-dimethylnona-2,4,6,8-tetraenoic acid) is a synthetic aromatic analogue of retinoic acid (Vitamin A derivative) indicated for the treatment of severe psoriasis, disorders of keratinisation and other dermatoses responsive to etretinate. Acitretin is an active metabolite of etretinate. Acitretin is available as oral capsules and tablets for systemic treatment. Acitretin is a known cause of birth defects when absorbed systemically. Acitretin was first developed in the 1970's by Hoffmann LaRoche Inc.

U.S. Pat. No. 4,105,681 describes a synthesis of acitretin.

b. Actinic Keratosis

Actinic keratosis (also called “solar keratosis” and “senile keratosis”) is a premalignant condition of thick, scaly, or crusty patches of skin. Actinic keratosis requires treatment, as in some cases it will progress to squamous cell carcinoma. Actinic keratosis is particularly suitable for topical treatment, as the lesions are usually relatively localized. Disadvantages with topical treatment may include skin irritation and low efficacy.

Existing topical treatments for actinic keratosis include imiquimod (marketed under the brand names Aldara and Zyclara), diclofenac (marketed as Solaraze) and 5-fluorouracil (Efudix).

c. Formulations

Current dosage forms of acitretin include oral tablets and capsules. The known oral dosage forms of acitretin result in the drug being absorbed systemically—that is, throughout the whole body. Systemic drug therapy has the disadvantage that the drug is distributed throughout the body's systems, not only where it is actually required. This may result in undesirable side effects in systems of the body other than those requiring treatment. For example, acitretin is known to cause birth defects in cases of in utero exposure.

It would therefore be advantageous, when using acitretin for the treatment or reduction of symptoms of dermatological conditions, to as far as possible confine the distribution of the drug to the skin—more particularly, to the area of skin requiring treatment, and to reduce or eliminate systemic absorption.

“Topical administration” refers to a drug or medication which is applied to a specific area of the skin of a subject and affects only or substantially only the area to which it is applied.

Topical use of acitretin has been suggested by Hsia et al. in “Effects of topically applied acitretin in reconstructed human epidermis and the rhino mouse”, J. Invest. Dermatol. 2008, Jan; 128(1):125-30. However, no commercial product has been released.

For a topical medicament to be effective it must be readily released from the vehicle matrix and interact intimately with the skin to be treated. In order to be effective, it is desirable for actives in topical compositions to be either fully dissolved or nano-sized, so as to achieve the necessary degree of penetration. However, this has proved difficult to achieve in the case of acitretin, in particular because the solubility characteristics of acitretin differ from other retinoids.

Typical solvents for use in creams would include alcohol or water. However, acitretin is in general very poorly soluble in water, so that an aqueous formulation is unlikely to be clinically efficacious. Acitretin is also quite poorly soluble in suitable alcohols. Also, it is undesirable to use large amounts of alcohol as a solvent in topical formulations, as the high levels of alcohol tend to irritate the skin of the user.

For example, U.S. Pat. No. 5,721,275 discloses topical compositions of retinoids in large concentrations of alcohol.

WO 2006/053006 proposes compositions comprising a retinoid, an anhydrous alcohol and an ester such as alkyl benzoate, isopropyl palmitate, diisopropyl adipate, or isopropyl myristate.

WO90/14833 describes aqueous gel vehicles for the topical application to the skin of irritating active ingredients such as retinoids, particularly tretinoin. The compositions include an aqueous medium, a gelling agent and an anti-oxidant. However, the amount of water in this formulation means it would be unsuitable for use with acitretin, which would be likely to crystallize. Also, the described formulations contain significant amounts of ethanol or isopropyl alcohol.

U.S. Pat. No. 4,034,114 describes a treatment to alleviate symptoms of keratosis consisting of topical compositions containing retinal. The compositions described contain significant amounts of alcoholic solvent and/or rely on solvents in which acitretin is much less soluble than is retinal.

U.S. Pat. No. 3,906,108 discloses a tretinoin cream emulsion for topical application which is stabilized by inclusion of xanthan gum. These formulations are ineffective in achieving and maintaining solubilization of acitretin.

It is therefore difficult to formulate a pharmaceutically acceptable topical cream containing acitretin. In particular, there is a strong tendency for acitretin to crystallize rather than remaining in solution in prior art formulations.

There remains a need for a topical acitretin composition with acceptable levels of efficacy and low irritancy.

### SUMMARY

The present invention is directed to improved topical compositions of acitretin for reducing at least one symptom of at least one dermatological condition, and to methods of manufacture and use of such compositions, in which acitretin is in the form of a nanosuspension.

In certain aspects, the compositions may be used to treat a subject, which may be a human subject or a mammal subject, diagnosed with a dermatological condition responsive to acitretin or etretinate or a symptom or symptoms of a dermatological condition responsive to acitretin or etretinate.

In certain aspects, the compositions may be used to treat a subject, which may be a human subject or a mammal subject, diagnosed with a keratinisation disorder, in particular actinic keratosis or with a symptom or symptoms of actinic keratosis.

In certain aspects the compositions are pharmaceutically acceptable formulations. In particular aspects, the compositions are gels. In certain particular aspects, the compositions may comprise a solid dispersion of acitretin in a copolymer of 1-vinyl-2-pyrrolidone and vinyl acetate in a ratio of 3:2 by mass (copovidone). A suitable copovidone copolymer is marketed under the trade mark Plasdone-S630.

The present invention is further directed to a topical medicament for reducing at least one symptom of at least one dermatological condition, and to methods of manufacture and use of such compositions, which comprises not less than 0.25% w/w acitretin, or at least about 0.5% w/w acitretin, and which shows a release rate of not less than 0.01 mg/cm² per min^{1/2} as measured using a Franz diffusion cell in vitro release testing system utilizing the following conditions: receptor medium comprising 1% DMSO in (35% ethanol:65% phosphate buffer pH 8.0), speed 700 rpm, membrane polysulfone 0.45 μm, dosage 300±30 mg, temperature 32.5±0.5° C.

In specific embodiments, the compositions of the invention may comprise acitretin which is in the form of a stable nanosuspension (as defined herein). The invention provides a topical medicament for reducing at least one symptom of at least one dermatological condition comprising acitretin particles as a nanosuspension, wherein at least 90%, by volume, of the acitretin particles suspended are 1 micron or less in size, and wherein at least 98%, by volume, of the acitretin particles suspended are 1 micron or less in size. The invention further provides the topical medicament wherein at least 99%, by volume, of the acitretin particles suspended are 1 micron or less in size. The invention further provides a topical medicament in gel form.

The invention further provides a topical medicament wherein the acitretin is a solid dispersion of acitretin with a copolymer. The invention further provides a topical medicament wherein acitretin is present at about 0.25-0.5% w/w. The invention further provides a topical medicament, wherein the copolymer is copovidone.

The invention further provides a topical medicament further comprising a dispersing agent, and further wherein the dispersing agent is a polysorbate, and further wherein the dispersing agent is polysorbate 20 present in an amount of less than about 0.3% w/w.

The invention further provides a topical medicament further comprising a chelating agent further wherein the chelating agent is EDTA. The invention further provides a topical medicament, wherein the composition comprises less than about 0.3% w/w polysorbate 20, and no EDTA. The invention further provides a topical medicament further comprising EDTA in the absence of polysorbate 20. The invention further provides a topical medicament further comprising EDTA in the presence of less than about 0.1% w/w polysorbate 20.

The invention further provides a topical medicament comprising residual solvent, further wherein the residual solvent is THF, and further wherein it is present in a concentration of at least about 0.4% w/w. The invention further provides a topical medicament, further comprising at least one preservative, further wherein the preservative is selected from the group consisting of a sodium paraben, sodium methylparaben, sodium propylparaben, potassium sorbate, phenoxyethanol, and combinations thereof.

The invention further provides a topical medicament further comprising propylene glycol of about 2.5% to about 5% w/w. The invention further provides a topical medicament wherein the composition comprises carbomer, further wherein acitretin is present at about 0.25-0.5 w/w, and the carbomer is between 0.4% and 0.6%.

The invention further provides a topical medicament wherein the medicament shows a release rate of not less than 0.01 mg/cm2 per min½ as measured using a Franz diffusion cell in vitro release testing system utilizing the following conditions: receptor medium comprising 1% DMSO in (35% ethanol: 65% phosphate buffer pH 8.0), speed 700 rpm, membrane polysulfone 0.45 μm, dosage 300±30 mg, temperature 32.5±0.5° C.

The invention provides a method of manufacture of the topical medicament which comprises forming a solid dispersion of acitretin particles and a copolymer of vinylpyrrolidone and vinyl acetate by spray drying pre-dissolved acitretin with a copolymer, and combining the solid dispersion with an aqueous gel base, further, wherein at least 90%, by volume, of the acitretin particles formed are 1 micron or less in size, further wherein at least 98%, by volume, of the acitretin particles formed are 1 micron or less in size, further wherein at least 99%, by volume, of the acitretin particles formed are 1 micron or less in size. The invention further provides the method wherein acitretin is present at about 0.25-0.5% w/w. The invention further provides the method wherein the copolymer is copovidone.

The invention further provides the method wherein the topical medicament further comprises a dispersing agent, wherein the dispersing agent is a polysorbate, further wherein the dispersing agent is polysorbate 20 present in an amount of less than about 0.3% w/w.

The invention further provides the method wherein the topical medicament further comprises a chelating agent, wherein the chelating agent is EDTA.

The invention further provides the method, wherein the composition comprises less than about 0.3% w/w polysorbate 20, and no EDTA. The invention further provides the method, further comprising EDTA in the absence of polysorbate 20. The invention further provides the method, further comprising EDTA in the presence of less than about 0.1% w/w polysorbate 20.

The invention further provides the method wherein the topical medicament comprises residual solvent, further wherein the residual solvent is THF, and further wherein it is present in a concentration of at least about 0.4% w/w.

The invention provides the method wherein the topical medicament further comprises at least one preservative, further wherein the preservative is selected from the group consisting of a sodium paraben, sodium methylparaben, sodium propylparaben, potassium sorbate, phenoxyethanol, and combinations thereof.

The invention provides the method wherein the topical medicament further comprises propylene glycol of about 2.5% to about 5% w/w. The invention further provides the method, wherein the topical medicament further comprises carbomer. The invention further provides the method wherein the topical medicament comprises acitretin at about 0.25-0.5% w/w, and the carbomer is between 0.4% and 0.6%.

The invention provides the method wherein the topical medicament shows a release rate of not less than 0.01 mg/cm2 per min½ as measured using a Franz diffusion cell in vitro release testing system utilizing the following conditions: receptor medium comprising 1% DMSO in (35% ethanol: 65% phosphate buffer pH 8.0), speed 700 rpm, membrane polysulfone 0.45 μm, dosage 300±30mg, temperature 32.5±0.5° C.

The foregoing and other objects, features and advantages of the present invention will become more readily apparent from the following detailed description of exemplary embodiments as disclosed herein.

### BRIEF DESCRIPTION OF THE DRAWINGS

The following figures form part of the present specification and are included to further demonstrate certain aspects of the present invention. The invention may be better understood by reference to one or more of the figures in combination with the detailed description of specific embodiments presented herein.

In the descriptions herein: “Triton X-100” is a trade mark for a product, the generic name for which is poly(oxy-1,2-ethanediyl), α[4-(1,1,3,3-tetramethylbutyl)phenyl]-ω-hydroxy; “Tween 20”: is a trade mark for a product, the generic term for which is polysorbate 20; and “Tween 80” is a trade mark for a product, the generic name for which is polysorbate 80.

Embodiments of the present invention are described, by way of example only, with reference to the attached figures, wherein:

FIG. 1A illustrates laser diffraction particle size distribution data for a sample of acitretin spray dried powder comprising 5% acitretin dispersed in 95% Plasdone -S630™ dispersed in water with 0.7% Triton X-100™.

FIG. 1B illustrates laser diffraction particle size distribution data for a sample of acitretin spray dried powder comprising 5% acitretin dispersed in 95% Plasdone -S630™, dispersed in water with 2% Tween 20™.

FIG. 1C illustrates laser diffraction particle size distribution data for a sample of acitretin spray dried powder comprising 5% acitretin dispersed in 95% Plasdone -S630™, dispersed in water with 2% Tween 80™.

FIG. 1D illustrates laser diffraction particle size distribution data for a sample of acitretin spray dried powder comprising 3% acitretin dispersed in 97% Plasdone -S630™, dispersed in water with 0.7% Triton X-100™.

FIG. 1E illustrates laser diffraction particle size distribution data for a sample of acitretin spray dried powder comprising 7.5% acitretin dispersed in 92.5% Plasdone -S630™, dispersed in water with 0.7% Triton X100™.

FIG. 1F illustrates laser diffraction particle size distribution data for a sample of acitretin spray dried powder comprising 10% acitretin dispersed in 90% Plasdone -S630™, dispersed in water with 0.7% Triton X100™.

FIG. 1G illustrates laser diffraction particle size distribution data for a sample of acitretin spray dried powder 12.5% acitretin dispersed in 87.5% Plasdone -S630™, dispersed in water with 0.7% Triton X100™.

FIG. 1H illustrates laser diffraction particle size distribution data for a sample of acitretin spray dried powder comprising 15% acitretin dispersed in 85% Plasdone -S630™, dispersed in water with 0.7% Triton X-100™.

FIG. 1I illustrates laser diffraction particle size distribution data for a sample of acitretin spray dried powder comprising 25% acitretin dispersed in 75% Plasdone -S630™, dispersed in water with 0.7% Triton X100™.

FIG. 2A illustrates the drug release profile, obtained by in vitro release testing using a Franz diffusion cell system as further described herein, for an acitretin gel formulation according to Example 2 herein.

FIG. 2B illustrates the drug release profile, obtained by in vitro release testing using a Franz diffusion cell system as further described herein, for an acitretin gel formulation according to Example 3 herein.

FIG. 2C illustrates the drug release profile, obtained by in vitro release testing using a Franz diffusion cell system as further described herein, for an acitretin gel formulation according to Example 4 herein.

FIG. 2D illustrates the drug release profile, obtained by in vitro release testing using a Franz diffusion cell system as further described herein, for an acitretin gel formulation according to Example 5 herein.

FIG. 2E illustrates the drug release profile, obtained by in vitro release testing using a Franz diffusion cell system as further described herein, for an acitretin gel formulation according to Example 6 herein.

FIG. 2F illustrates the drug release profile, obtained by in vitro release testing using a Franz diffusion cell system as further described herein, for an acitretin gel formulation according to Example 7 herein.

FIG. 2G illustrates the drug release profile, obtained by in vitro release testing using a Franz diffusion cell system as further described herein, for an acitretin gel formulation according to Example 8 herein.

FIG. 2H illustrates the drug release profile, obtained by in vitro release testing using a Franz diffusion cell system as further described herein, for an acitretin gel formulation according to Example 9 herein.

FIG. 2I is a bar chart presentation of the average release rate of acitretin gel formulations according to Examples 2-9 herein.

FIG. 3A shows an optical microscopic image of spray dried acitretin solid dispersion (5% acitretin dispersed in 95% Plasdone -S630™) at 400× magnification.

FIG. 3B shows an optical microscopic image of a sample of a gel preparation containing spray dried acitretin solid dispersion, the solid dispersion comprising 5% w/w acitretin dispersed in 95% Plasdone -S630™ shortly after the time of preparation, at 1000× magnification.

FIG. 3C shows an optical microscopic image of the sample in FIG. 3B after 14 days storage at 40° C./75% RH, at 1000× magnification.

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, methods, procedures and components have not been described in detail so as not to obscure the embodiments described herein.

The present invention is directed to systems, methods and compositions for the topical administration of acitretin.

In exemplary embodiments, a subject in need of treatment for one or more dermatological conditions or signs or symptoms of one or more dermatological conditions, such as a mammal, and in specific embodiments a human, is administered acitretin topically. In such embodiments, the one or more dermatological conditions may include actinic keratosis.

In specific embodiments, the subject in need of treatment is a subject exhibiting one or more signs or symptoms of actinic keratosis. In such embodiments, signs or symptoms may include one or more of the following: precancerous or premalignant flat or thickened, scaly, warty or horny, skin coloured or reddened lesions.

In specific embodiments, the compositions of the invention may be pharmaceutical compositions in which acitretin is in the form of a stable nanosuspension. By “stable” is meant at least 90% of potency of the drug substance is preserved during at least 3 months storage at 40° C./75% RH without significant change in the rate and extent to which the drug product is released from the product matrix. In certain particularly preferred embodiments longer stability may be observed, for example at least 90% of potency of the drug substance may be preserved during at least 6 months storage at 40° C./75% RH and/or at least 9 months or at least 12 months storage at 25° C./60% RH without significant change in the rate and extent to which the drug product is released from the product matrix. By “significant change” is meant more than about 10-15% change.

The amount of acitretin in the present compositions will depend on the particular application. Generally topical acitretin compositions in accordance with this invention may contain, for example, from 0.01 to 1% w/w acitretin. In specific embodiments compositions in accordance with this invention may, for example, contain 0.03%, 0.05%, 0.1%, 0.15%, 0.2%, 0.25%, 0.3%, 0.35%, 0.4%, 0.45%, 0.5%, 0.6% or 0.75% acitretin on a weight basis. The precise amount of acitretin may in part be chosen to optimize the desired release rate.

Gels

In specific embodiments, the compositions of the invention may be formulated as a gel. By a “gel” is meant a pharmaceutical preparation comprising a colloid in which a solid dispersed phase forms a network in combination with a fluid continuous phase, resulting in a viscous semirigid solid.

In specific embodiments, the present invention discloses gels in which acitretin is present as a substantially stable nanosuspension. By “nanosuspension” is meant a preparation in which nano sized solid acitretin is dispersed in a liquid phase. The acitretin may be amorphous.

In specific embodiments, the gels of the invention may further comprise copovidone. Copovidone is a copolymer of 1-vinyl-2-pyrrolidone and vinyl acetate.

In specific embodiments, the present invention discloses a method of manufacture of a stable nanosuspension of acitretin, which comprises forming a solid dispersion of acitretin with a copolymer, preferably by spray drying pre-dissolved acitretin with copovidone (a copolymer of 1-vinyl-2-pyrrolidone and vinyl acetate), and combining the resulting powder with an aqueous gel base. By a “solid dispersion” is meant a solid material in which the active is dispersed in an amorphous state. This may result for example from the active being fully solubilized in a solvent, such as tetrahydrofuran (THF), before being spray dried with the copolymer. FIG. 4A shows an optical microscopic image of such a solid dispersion.

When mixed with the aqueous gel base, the spray dried powder particles are seen microscopically as homogeneous spheres of approximately 5 to 50 microns in diameter comprising acitretin dispersed in copovidone. FIG. 4B shows such a gel preparation immediately after addition of the spray dried solid dispersion. It has been surprisingly observed that over the course of less than around 24 hours, in certain preferred embodiments less than 1 hour, the spheres dissolve resulting in a gel matrix containing very small (sub-micron) precipitated particles of acitretin with a relatively uniform particle size distribution within the gel matrix. The particle size distribution determined by laser diffraction indicates that the majority of the acitretin particles are less than 1 micron in size. In some instances, although the particle size is small, some agglomeration of the particles may initially occur. A brief application of sonication, for example a 30 second internal pulse of sonication, may be required to disperse such agglomerates and allow the true particle size to be determined. By optical microscopy, as shown by FIG. 4C, the precipitated acitretin particles can be seen to be surprisingly homogeneous in size and shape. A particle size distribution where the majority of the particles are less than 1 micron in size can improve the topical absorption of insoluble drug substances such as acitretin.

For example, in particular embodiments, at least 90% or at least 98%, or at least 99% of the acitretin particles suspended in the gel (on a volume basis) are 1 micron or less in size (i.e., D(v,0.90)NMT 1 micron). It has been found that acitretin spray dried powder exhibiting a particle size value d90>1 micron when dispersed in 0.7% Triton-X, does not effectively form a nanosuspension within the topical gel composition of the invention.

The co-precipitation of polymer and active in various ratios produces solid dispersions. In specific embodiments, the spray dried powder comprises about 5% acitretin and about 95% copovidone (w/w). In certain embodiments, the ratio of % acitretin to % copovidone in the spray dried powder may be less than 50:50, in particular less than or equal to 25:75. In certain embodiments, the ratio of % acitretin to % copovidone in the spray dried powder may be 25:75, or 20:80, or 15:85, or 12.5:87.5, or 10:90, or 7.5:92.5, or 3:97.

In general, it has been found that acitretin spray dried powders having between 3% and 25% acitretin can be used to formulate a topical gel composition containing a nanosuspension of acitretin in accordance with the invention. By contrast, gels prepared using micronized acitretin capsule fill (not spray dried powder), do not result in nanosuspension.

In certain specific embodiments, gel formulations according to the invention may comprise a suitable dispersing agent. For example, a suitable dispersing agent may be a polysorbate, for example polysorbate 20, which is sold under the brand name Tween 20™.

If Tween 20 is added, then it may be necessary to avoid the use of effective chelators, such as sodium edentate (EDTA). EDTA is commonly used as a manufacturing and preserving agent. However, it has been found that as an effective chelator, EDTA can promote crystallization of acitretin in the compositions of the invention, when the acitretin is not contained within the spray dried powder spheres.

At levels above about 0.3% w/w, it has been found that Tween 20 can partially dissolve the acitretin nano-particles. Over time, the dissolved portion of the acitretin is susceptible to spontaneous recrystallisation. When this occurs it can promote further dissolution and recrystallisation of acitretin as relatively large, typically >1 micron, acitretin crystals. In the presence of EDTA, the dissolved acitretin will relatively rapidly form and grow crystals. In the absence of EDTA, the escaped acitretin resulting from inclusion of Tween 20 levels greater than 0.3% w/w will slowly grow crystals. In the absence of Tween 20, a nanodispersed acitretin gel containing EDTA exhibits no crystal growth.

Therefore, preferred formulations according to the invention may contain less than about 0.3% w/w Tween 20, and preferably no EDTA, or may contain EDTA in the absence of Tween 20, or in the presence of only very low levels of Tween 20, for example less than about 0.1% w/w.

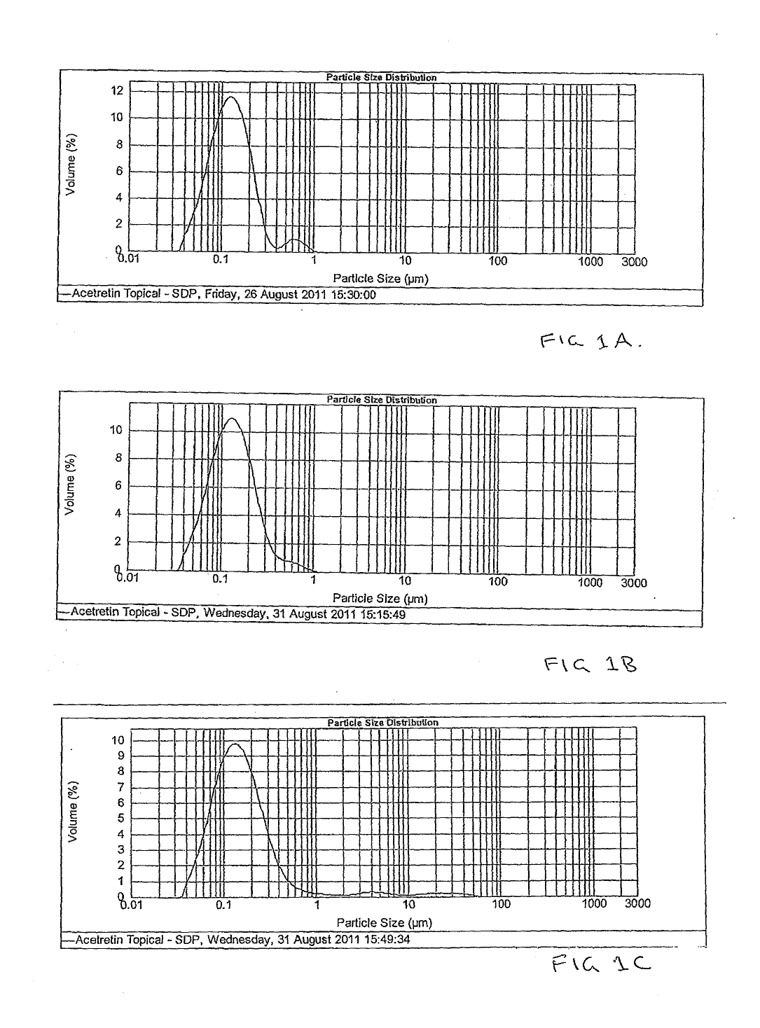

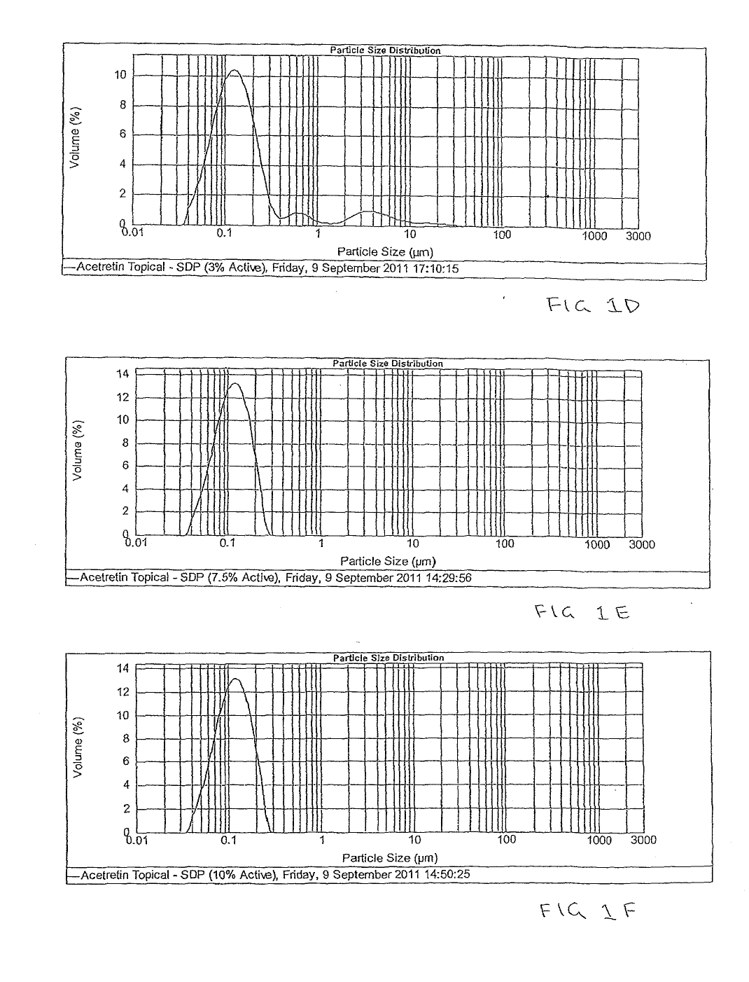

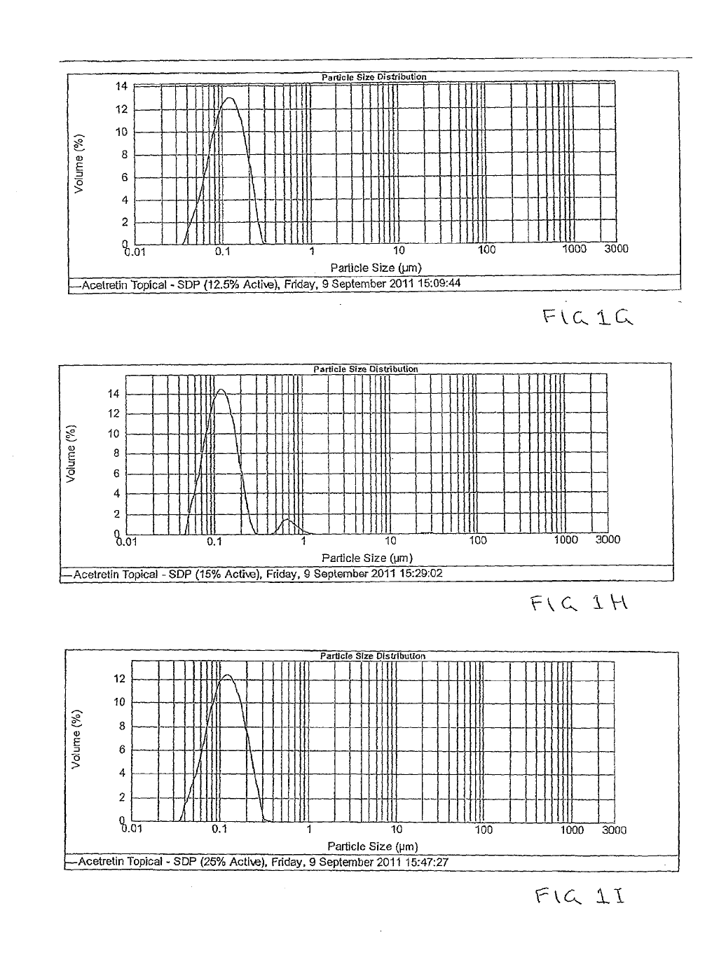

FIGS. 1A to 1I show the results of particle size distribution analysis for samples of acitretin spray dried powder (containing a range of ratios of acitretin in copovidone) dispersed in water together with a nonionic surfactant or dispersant, which in these examples is either 0.7% Triton X-100 or 2% Tween 20 or 80. Each of these examples shows a D(0.9) of less than one micron.

The spray dried powder containing acitretin active typically also contains residual solvent such as THF. It has been found that if the residual THF content of the spray-dried powder falls below about 0.4% w/w, the ability of the acitretin in the spray dried powder to yield nano-dispersions during gel formulation is lost. Instead, the acitretin in the spray dried powder tends to aggregate to form large crystals and does not form a nanosuspension when formulated into a gel.

Therefore, it is preferred that the residual THF content of the acitretin spray dried powder is 0.4% w/w or above. However, when determining the acceptable residual level, it is also necessary to take into account the intended daily dosage of the composition in use, when compared with the permitted daily exposure limit for THF.

In certain preferred embodiments the gel formulation according to the invention may include one or more preservatives. Suitable preservatives include sodium parabens, such as sodium methylparaben or sodium propylparaben, potassium sorbate, and phenoxyethanol. These ingredients can be used either singularly or in combination of two or more compounds. The exact levels of particular preservatives will be determined in order to achieve desired levels of preservative efficacy in particular instances.

It has been found that interactions between preservatives in the composition can affect the successful formation of a nanosuspension. In particular it appears that the sodium parabens can play an additional role in generating a nanosuspension, over and above simply raising the pH. For example, if potassium sorbate or phenoxyethanol are present without sodium methylparaben and sodium propylparaben also being present, the nanosuspension tends not to form, even with the use of sodium hydroxide to raise the pH. However, a nanosuspension will form when potassium sorbate or phenoxyethanol are used as preservatives in combination with sodium methylparaben or sodium propylparaben.

Other possible excipients may be utilized in the formulation. For example, propylene glycol may help to preserve the formulation. The level of propylene glycol used will affect the viscosity of the formulation. For example at 10% w/w propylene glycol the gel is quite runny. A preferred level of propylene glycol is about 2.5% to about 5%, most preferably about 5%.

In certain preferred embodiments the gel formulation according to the invention may contain carbomer. Suitable carbomers include high molecular weight crosslinked polymers of acrylic acid, for example Carbomer 974P. The level of carbomer should be chosen so as to achieve a suitable viscosity and an IVRT release rate for a 0.25-0.5 w/w acitretin gel of not less than about 0.010 mg/cm²/min^{1/2}. For example, for a 0.25-0.5% w/w acitretin gel a suitable level of carbomer is between 0.4% and 0.6%, more preferably 0.45-0.5%, most preferably about 0.45%.

According to at least one presently preferred embodiment of the invention a stable topical gel formulation comprising 0.5% w/w acitretin as a stable nanosuspension displaying a release rate of not less than about 0.01 mg/cm² may comprise 2.50% acitretin spray dried powder containing 1:4 acitretin:copovidone, 0.3-0.8%, preferably 0.4-0.5%, most preferably 0.45% carbomer 974P, 1.0-10%, preferably 2.5-7.5%, most preferably 5.0% propylene glycol, up to 0.40%, preferably about 0.20% sodium methylparaben, up to 0.73%, preferably about 0.40% sodium propylparaben, about 7.50% copovidone filler, and water.

According to at least one further presently preferred embodiment of the invention a stable topical gel formulation comprising 0.5% w/w acitretin as a stable nanosuspension displaying a release rate of not less than about 0.01 mg/cm² may comprise 10.00% acitretin spray dried powder containing 1:19 acitretin:copovidone, 0.3-0.8%, preferably 0.4-0.5%, most preferably 0.45% carbomer 974P, 1.0-10%, preferably 2.5-7.5%, most preferably 5.0% propylene glycol, up to 0.40%, preferably about 0.20% sodium methylparaben, up to 0.73%, preferably about 0.40% sodium propylparaben, and water.

According to at least one further presently preferred embodiment of the invention a stable topical gel formulation comprising 0.25% w/w acitretin as a stable nanosuspension may comprise 1.25% acitretin spray dried powder containing 1:4 acitretin:copovidone, 0.3-0.8%, preferably 0.4-0.7%, most preferably 0.50% carbomer 974P, 1.0-10%, preferably 2.5-7.5%, most preferably 5.0% propylene glycol, up to 0.40%, preferably about 0.20% sodium methylparaben, up to 0.73%, preferably about 0.40% sodium propylparaben, about 8.50% copovidone filler, and water.

According to at least one further presently preferred embodiment of the invention a stable topical gel formulation comprising 0.25% w/w acitretin as a stable nanosuspension may comprise 5.00% acitretin spray dried powder containing 1:19 acitretin:copovidone, 0.3-0.8%, preferably 0.4-0.7%, most preferably 0.50% carbomer 974P, 1.0-10%, preferably 2.5-7.5%, most preferably 5.0% propylene glycol, up to 0.40%, preferably about 0.20% sodium methylparaben, up to 0.73%, preferably about 0.40% sodium propylparaben, about 4.75% copovidone filler, and water.

According to at least one further presently preferred embodiment of the invention a stable topical gel formulation comprising 0.25% w/w acitretin as a stable nanosuspension displaying a release rate of not less than about 0.01 mg/cm² may comprise 1.25% acitretin spray dried powder containing 1:4 acitretin:copovidone, about 0.50% carbomer 974P, 1.0-10%, preferably 2.5-7.5%, most preferably 5.0% propylene glycol, up to 0.40%, preferably about 0.20% sodium methylparaben, up to 0.73%, preferably about 0.40% sodium propylparaben, about 8.50% copovidone filler, and water.

Dosage

The actual dosage amount of a composition for delivery of drugs can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

An effective amount of the therapeutic composition is determined based on the intended goal. As a topical composition, the compositions of the invention are intended to be applied directly to the affected area or lesion, for example with a fingertip. The quantity to be administered, both according to number of treatments and unit dose, depends on the protection or effect desired.

Packaging

The compositions of the invention may be packaged for use in various forms of packaging for gels as are known in the art. For example, the gel may be packaged in a tube, such as an aluminium barrier laminate tube, having a relatively large diameter orifice, for example around 8 mm, in which case a relatively viscous product (for example, containing 0.6% carbomer, as in Example 8) may be desirable to prevent leakage. Alternatively, the gel may be packaged in a small orifice container, a pump or sachet, in which case a less viscous (i.e. runnier) formulation may be more suitable (for example containing 0.4% carbomer, as in Example 9) .

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice.

However, those of skill in the art should, in light of the present invention, appreciate that many changes can be made in the specific embodiments which are disclosed and still obtain a like or similar result without departing from the spirit or scope of the invention. The following examples are offered by way of illustration and not by way of limitation.

### Example 1A

### Preparation of Amorphous Acitretin 5% w/w Spray Dried Powder

| Ingredient | Mass (g) | Amount (% w/w) |
|---|---|---|
| Acitretin | 5 | 0.83 |
| Copovidone | 95 | 15.83 |
| Tetrahydrofuran | 500 | 83.34 |
| | Total | 100.00 |

1. Dissolve the copovidone and acitretin in THF with constant stirring.

2. Spray dry the resulting solution using a co-current two fluid nozzle under an atmosphere of nitrogen process gas with an inlet temperature of 120° C. and an exhaust temperature of 80° C.

### Example 1B

### Preparation of Amorphous Acitretin 20% w/w Spray Dried Powder

| Ingredient | Mass (g) | Amount (% w/w) |
|---|---|---|
| Acitretin | 300 | 1.82 |
| Copovidone | 1200 | 7.27 |
| Tetrahydrofuran | 15000 | 90.91 |
| | Total | 100.00 |

1. Dissolve the copovidone and acitretin in THF with constant stirring.

2. Spray dry the resulting solution using a co-current two fluid nozzle under an atmosphere of nitrogen process gas with an inlet temperature of 120° C. and an exhaust temperature of 80° C.

### Example 2

### 0.5% w/w Acitretin Gel Formulation

| Ingredient | Mass (g) | Amount in product (% w/w) |
|---|---|---|
| Carbomer 974P | 0.45 | 0.45 |
| Propylen glycol | 4.5 | 4.5 |
| EDTA | 0.09 | 0.09 |
| Sodium Methylparaben | 0.27 | 0.27 |
| Sodium Propylparaben | 0.18 | 0.18 |
| Amorphous Acitretin Spray | 10.0 | 10.00 |
| Dried Powder (5% acitretin, 95% | | |
| copovidone) | | |
| Water | 84.51 | 84.51 |
| Total | 100.0 | 100.0 |

Gel Preparation

1. Stir the water with open-blade impeller overhead mixer at 1000 rpm, generating a vortex slightly larger than the impeller diameter.

2. Slowly sprinkle the carbomer into the vortex, followed by all the other excipients except the amorphous spray dried powder comprising 5% w/w acitretin and 95% w/w copovidone.

3. Continue mixing at a reduced speed until the mixture appears homogeneous.

4. Into a 500 mL beaker weigh amorphous acitretin spray dried powder (5% acitretin, 95% copovidone as a solid dispersion) 10.0 g

5. Carefully transfer the gel into the beaker containing amorphous acitretin and mix with an overhead mixer to obtain a smooth gel.

6. Stir carefully, knocking, tapping, scraping the sides of the beaker to aid homogenisation.

This example formulation was found to work well, with no crystal formation.

### Example 3

### 0.25% w/w Acitretin Gel Formulation

| Ingredient | Mass (g) | Amount in product (% w/w) |
|---|---|---|
| Carbomer 974P | 5.00 | 0.50 |
| Propylene Glycol | 50.00 | 5.00 |
| BHT | 1.00 | 0.10 |
| Sodium Methylparaben | 2.50 | 0.25 |
| Sodium Propylparaben | 1.50 | 0.15 |
| Amorphous Acitretin Spray | 50.00 | 5.00 |
| Dried Powder (5% acitretin, 95% | | |
| copovidone) | | |
| Copovidone | 47.50 | 4.75 |
| Water | 842.5 | 84.25 |
| Total | 1000.0 | 100.0 |

Gel Preparation

1. Stir the water (840.0 g)in a 2 L beaker with two-tier paddle on overhead mixer at high speed, generating a strong vortex.

2. Sprinkle the carbomer into the vortex over period of 10 minutes to avoid clumping.

3. Add the BHT and continue stirring for another 40 minutes on reduced vortex to dissolve the carbomer.

4. Add the propylene glycol.

5. Add the Sodium methylparaben. To aid efficient mixing increase the rpm as the gel thickens.

6. Add the Sodium propylparaben.

7. Continue mixing at high speed until homogeneous. Spatula the sides if necessary.

8. Split the gel into two equal amounts.

9. To one of the two parts add amorphous spray dried powder comprising 5% w/w acitretin and 95% w/w copovidone and stir with an overhead mixer.

10. To the second portion of the gel add the copovidone and water (2.5 g) and stir with an overhead mixer.

11. Combine and mix together the gels from Steps 9 and 10.

This example formulation was found to work well, with no crystal formation.

### Example 4

### 0.5% w/w Acitretin Gel Formulation with 0.3% w/w Tween 20

| Ingredient | Mass (g) | Amount % (w/w) |
|---|---|---|
| Water (I) | 6553.0 | 81.67 |
| Carbomer 974 | 48.0 | 0.60 |
| Water (II) | 100.0 | 1.25 |
| Sodium methylparaben | 16.0 | 0.20 |
| Sodium propylparaben | 32.0 | 0.40 |
| Propylene Glycol | 400.0 | 5.00 |
| Amorphous Acitretin SDP (4.7%) | 851.0 | 10.61 |
| Post manufacture spike with Tween 20 | 24.0 | 0.30 |
| Total | 8024.0 | 100 |

Gel Preparation

1. Using a large overhead mixer with a three-pronged paddle, stir water (I) in a 1 L beaker with an overhead mixer at high speed, generating a strong vortex.

2. Sprinkle the carbomer into the water over period of 10 minutes to avoid clumping. Continue stirring until carbomer has fully hydrated, free from any lumps.

3. Into a small vessel fully dissolve the parabens into water (II) with a bench top overhead mixer.

4. Into a medium vessel dispense propylene glycol.

5. Into the propylene glycol from step 4 admix the parabens solution from step 3.

6. Into the carbomer solution from step 2 admix the glycolic parabens solution from step 5, increasing the mixing speed as the gel cures.

7. While stirring at high speed with the large overhead mixer, add the Amorphous Acitretin SDP to the cured base gel.

8. Continue mixing and observe the gel under microscope until the gel exhibits uniform homogeneity of nanodispersions.

9. Add the Tween 20 to the gel from step 8. Mix the sample well with low shear overhead mixer accurately for 5 minutes.

### Example 5

### 0.5% w/w Acitretin Gel Formulation using Acitretin 20% w/w Spray Dried Powder

| Ingredient | Mass (kg) | Amount % (w/w) |
|---|---|---|
| Water (I) | 16.360 | 81.80 |
| Copovidone | 1.480 | 7.40 |
| Carbomer 974 | 0.120 | 0.60 |
| Amorphous Acitretin SDP (19.4 %) | 0.335 | 1.68 |
| Amorphous Acitretin SDP (19.1 %) | 0.184 | 0.92 |
| Water (II) | 0.400 | 2.00 |
| Sodium methylparaben | 0.040 | 0.20 |
| Sodium propylparaben | 0.080 | 0.40 |
| Propylene Glycol | 1.000 | 5.00 |
| Total | 20.00 | 100.0 |

Gel Preparation

1. Into a 50 L medicine tank chamber, homogenize/mix water (I).

2. Sprinkle the carbomer into the water (I) over period of 10 minutes to avoid clumping. Continue homogenizing/mixing until carbomer has fully hydrated, free from any lumps.

3. Add the copovidone while homogenizing/mixing.

4. Add all the Amorphous Acitretin SDP while homogenizing/mixing.

5. Into a medium vessel fully dissolve the parabens in water (II) using a bench top overhead mixer.

6. Into the parabens solution admix the propylene glycol.

7. Into the medicine tank add the glycolic parabens solution to cure the gel, mixing with paddle mixer only.

8. Continue mixing and observe the gel under microscope until the gel exhibits uniform homogeneity of nanosuspension.

### Example 6

### 0.25% w/w Acitretin Gel Formulation using Acitretin 20% w/w Spray Dried Powder

| Ingredient | Mass (kg) | Amount % (w/w) |
|---|---|---|
| Water (I) | 16.410 | 82.05 |
| Copovidone | 1.691 | 8.46 |
| Carbomer 974 | 0.120 | 0.60 |
| Amorphous Acitretin SDP (19.3%) | 0.259 | 1.30 |
| Water (II) | 0.400 | 2.00 |
| Sodium methylparaben | 0.040 | 0.20 |
| Sodium propylparaben | 0.080 | 0.40 |
| Propylene Glycol | 1.000 | 5.00 |
| Total | 20.000 | 100.0 |

Gel Preparation

1. Into a 50 L medicine tank chamber, homogenize/mix water (I).

2. Sprinkle the carbomer into the water (I) over period of 10 minutes to avoid clumping. Continue homogenizing/mixing until carbomer has fully hydrated, free from any lumps.

3. Add the copovidone while homogenizing/mixing.

4. Add Amorphous Acitretin SDP while homogenizing/mixing.

5. Into a medium vessel fully dissolve the parabens in water (II) using a bench top overhead mixer.

6. Into the parabens solution admix the propylene glycol.

7. Into the medicine tank add the glycolic parabens solution to cure the gel, mixing with paddle mixer only.

8. Continue mixing and observe the gel under microscope until the gel exhibits uniform homogeneity of nanosuspension.

### Example 7

### 0.5% w/w Acitretin Gel Formulation with 0.6% w/w Carbomer

| Ingredient | Mass (g) | Amount % (w/w) |
|---|---|---|
| Water (I) | 400.0 | 80.0 |
| Carbomer 974 | 3.0 | 0.6 |
| Copovidone | 37.5 | 7.5 |
| Amorphous Acitretin SDP | 12.5 | 2.5 |
| (20:80 API:Plasdone) | (2.5:10) | (0.5:2.0) |
| Propylene Glycol | 25.0 | 5.0 |
| Sodium Methylparaben | 1.0 | 0.2 |
| Sodium Propylparaben | 2.0 | 0.4 |
| Water (II) | 10.0 | 2.0 |
| Water (III) | 9.0 | 1.8 |
| Total | 500.0 | 100.0 |

Gel Preparation

1. Stir water (I) in a 1 L beaker with an overhead mixer at high speed, generating a strong vortex.

2. Sprinkle the carbomer into the water slowly to avoid clumping. Continue stirring until carbomer is fully hydrated.

3. While stirring to generate a vortex, add the copovidone to the hydrated carbomer.

4. While stirring to generate a vortex, add the Acitretin SDP to the hydrated carbomer.

5. Dissolve the sodium methylparaben and sodium propylparaben in water (II) in a small beaker.

6. Into a small beaker containing the propylene glycol stir-in the paraben solution.

7. Add the glycolic paraben mix to the carbomer solution while stirring, increasing the mixing speed as the gel thickens. Continue mixing until homogeneous.

8. Add Water (III) with stirring to achieve a net mass of 500 g.

### Example 8

### 0.5% w/w Acitretin Gel Formulation with 0.4% w/w Carbomer

| Ingredient | Mass (g) | Amount % (w/w) |
|---|---|---|
| Water (I) | 400.0 | 80.0 |
| Carbomer 974 | 2.0 | 0.4 |
| Copovidone | 37.5 | 7.5 |
| Amorphous Acitretin | 12.5 | 2.5 |
| (20:80 API:Plasdone) | (2.5:10) | (0.5:2.0) |
| Propylene Glycol | 24.5 | 4.9 |
| Sodium Methylparaben | 1.0 | 0.2 |
| Sodium Propylparaben | 2.0 | 0.4 |
| Water (II) | 10.0 | 2.0 |
| Water (III) | 10.5 | 2.1 |
| Total | 500.0 | 100.0 |

Gel Preparation

1. Stir water (I) in a 1 L beaker with an overhead mixer at high speed, generating a strong vortex.

2. Sprinkle the carbomer into the water slowly to avoid clumping. Continue stirring until carbomer is fully hydrated.

3. While stirring to generate a vortex, add the copovidone to the hydrated carbomer.

4. While stirring to generate a vortex, add the Acitretin SDP to the hydrated carbomer.

5. Dissolve the sodium methylparaben and sodium propylparaben in water (II) in a small beaker.

6. Into a small beaker containing the propylene glycol stir-in the paraben solution.

7. Add the glycolic paraben mix to the carbomer solution while stirring, increasing the mixing speed as the gel thickens. Continue mixing until homogeneous.

8. Add Water (III) with stirring to achieve a net mass of 500 g.

### Example 9

### 0.5% w/w Acitretin Gel Formulation with 0.45% w/w Carbomer

| Ingredient | Mass (g) | Amount % (w/w) |
|---|---|---|
| Water (I) | 400.0 | 80.00 |
| Carbomer 974 | 2.25 | 0.45 |
| Propylene Glycol | 22.50 | 4.50 |
| Sodium methylparaben | 1.00 | 0.20 |
| Sodium propylparaben | 2.00 | 0.40 |
| Amorphous Acitretin SPD | 50.00 | 10.00 |
| (5% acitretin, 95% plasdone) | | |
| Water (II) | 22.25 | 4.45 |
| Total | 500.0 | 100.0 |

Gel Preparation

1. Using the two-tier paddle stir 400 g water in a 1 L beaker with an overhead mixer at high speed, generating a strong vortex.

2. Sprinkle the carbomer into the vortex over period of 10 minutes to avoid clumping. Continue stirring until mixture has thickened.

3. Sequentially add the other base gel excipients, increasing the mixing speed as the gel thickens. Continue mixing until homogeneous

4. Increase/adjust the mixing speed to obtain and maintain a small vortex as the Acitretin spray-dried powder is slowly spooned into the vortex. Stir carefully tap and scrape the sides of the beaker to aid homogenisation.

5. Add Water (II) with stirring to achieve a net mass of 500 g.

Efficacy—In Vitro Release testing

For a topical medicament to be effective it must be readily released from the vehicle matrix and interact intimately with the skin to be treated. On this basis candidate formulations can be ranked based on in vitro release rates through artificial or post mortem skin membranes. This is routinely undertaken using the Franz Diffusion Cell methodology. The rate and extent to which the drug substance is released from the product matrix are particularly relevant to the prediction of relative efficacy of candidate formulations.

In Vitro Release Testing (IVRT) is a useful test to assess product “sameness” under certain scale and post approval changes for semisolid products. The FDA Guidance on Scale up and Post Approval Changes for Semisolid (SUPAC-SS) describes suitable conditions for this testing.

The apparatus used for IVRT is a Franz diffusion cell system acquired from Hanson Research. It consists of six individual cells. Each cell has a standard open cap ground glass surface with 15 mm diameter orifices, 7 mL volume capacity, and total diameter of 25 mm. About 300 mg of the semisolid preparation is placed uniformly on a synthetic membrane and kept occluded to prevent solvent evaporation and compositional changes. Multiple sampling times (at least 5 times) over an appropriate time period are suggested in order to generate an adequate release profile and to determine the drug release rate.

The conditions used for IVRT of the example formulations of the invention are as follows:

| | |
|---|---|
| Receptor | 1% DMSO in (35% Ethanol: |
| Medium | 65% phosphate buffer pH 8.0) |
| Speed | 700 rpm |
| Membrane | Pall Life Sciences Tuffryn ® Polysulfone 0.45 μm |
| Dosage | 300 ± 30 mg |
| Temperature | 32.5 ± 0.5° C. |

The following table shows the results of IVRT under the above conditions on Acitretin 0.5% w/w Gel (Example 2)—:

### Example 2

### Acitretin 0.5% w/w Gel

| | Cell 1 | Cell 2 | Cell 3 | Cell 4 | Cell 5 | Cell 6 |
|---|---|---|---|---|---|---|
| Total Release, | 24.6 | 23.4 | 22.7 | 22.5 | 21.7 | 22.3 |
| % | | | | | | |
| Release Rate | 0.015 | 0.0130 | 0.014 | 0.014 | 0.013 | 0.013 |
| (mg/cm² | | | | | | |
| per min^{1/2}) | | | | | | |
| Regression (r) | 0.9909 | 0.9857 | 0.9835 | 0.9832 | 0.9826 | 0.9819 |

Average total release = 22.9%
Average release rate = 0.014 (RSD = 6.0%)
Average Regression = 0.9846

The following table shows the results of IVRT under the above conditions on Acitretin 0.25% w/w Gel (Example 3)—

### Example 3

### Acitretin 0.25% w/w Gel

| | Cell 1 | Cell 2 | Cell 3 | Cell 4 | Cell 5 | Cell 6 |
|---|---|---|---|---|---|---|
| Total Release, | 34.7 | 31.5 | 27.3 | 36.0 | 28.8 | 28.6 |
| % | | | | | | |
| Release Rate | 0.011 | 0.010 | 0.009 | 0.011 | 0.010 | 0.010 |
| (mg/cm² | | | | | | |
| per min^{1/2}) | | | | | | |
| Regression (r) | 0.9767 | 0.9769 | 0.9718 | 0.9797 | 0.9758 | 0.9796 |

Average total release = 31.1%
Average release rate = 0.010 (RSD = 7.4%)
Average Regression = 0.9763

The following table shows the results of IVRT under the above conditions on Acitretin 0.5% w/w Gel (Example 4)

### Example 4

### Acitretin 0.5% w/w GEL

| | Cell 1 | Cell 2 | Cell 3 | Cell 4 | Cell 5 | Cell 6 |
|---|---|---|---|---|---|---|
| Total Release, | 19.0 | 17.6 | 16.5 | 17.6 | 19.6 | 16.3 |
| % | | | | | | |
| Release Rate | 0.012 | 0.011 | 0.010 | 0.011 | 0.012 | 0.010 |
| (mg/cm² | | | | | | |
| per min^{1/2}) | | | | | | |
| Regression (r) | 0.9776 | 0.9773 | 0.9751 | 0.9757 | 0.9719 | 0.9784 |

Average total release = 17.8%
Average release rate = 0.011 mg/cm²/min^{1/2} (RSD = 7.7%)
Average Regression = 0.9760

The following table shows the results of IVRT under the above conditions on Acitretin 0.5% w/w Gel (Example 5)

### Example 5

### Acitretin 0.5% w/w GEL,

| | Cell 1 | Cell 2 | Cell 3 | Cell 4 | Cell 5 | Cell 6 |
|---|---|---|---|---|---|---|
| Total Release, | 20.5 | 18.7 | 18.4 | 15.7 | 17.9 | 17.2 |
| % | | | | | | |
| Release Rate | 0.013 | 0.012 | 0.012 | 0.010 | 0.012 | 0.012 |
| (mg/cm² | | | | | | |
| per min^{1/2}) | | | | | | |
| Regression (r) | 0.9811 | 0.9768 | 0.9723 | 0.9773 | 0.9716 | 0.9698 |

Average total release = 18.1%
Average release rate = 0.012 mg/cm²/min^{1/2} (RSD = 8.3%)
Average Regression = 0.9748

The following table shows the results of IVRT under the above conditions on Acitretin 0.25% w/w Gel (Example 6).

### Example 6

### Acitretin 0.25% w/w Gel

| | Cell 1 | Cell 2 | Cell 3 | Cell 4 | Cell 5 | Cell 6 |
|---|---|---|---|---|---|---|
| Total Release, | 23.8 | 25.3 | 23.7 | 22.2 | 21.8 | 21.4 |
| % | | | | | | |
| Release Rate | 0.008 | 0.008 | 0.007 | 0.007 | 0.007 | 0.007 |
| (mg/cm² | | | | | | |
| per min^{1/2}) | | | | | | |
| Regression (r) | 0.9801 | 0.9728 | 0.9779 | 0.9796 | 0.9758 | 0.9774 |

Average total release = 23.0%
Average release rate = 0.007 mg/cm²/min^{1/2} (RSD = 7.0%)
Average Regression = 0.9773

The following table shows the results of IVRT under the above conditions on Acitretin 0.5% w/w Gel (Example 7).

### Example 7

### Acitretin 0.5% w/w Gel

| | Cell 1 | Cell 2 | Cell 3 | Cell 4 | Cell 5 | Cell 6 |
|---|---|---|---|---|---|---|
| Total Release, | 20.0 | 19.8 | 18.8 | 18.1 | 19.6 | 16.8 |
| % | | | | | | |
| Release Rate | 0.013 | 0.012 | 0.012 | 0.012 | 0.012 | 0.011 |
| (mg/cm² | | | | | | |
| per min^{1/2}) | | | | | | |
| Regression (r) | 0.9807 | 0.9741 | 0.9723 | 0.9772 | 0.9819 | 0.9800 |

Average total release = 18.9%
Average release rate = 0.012 mg/cm²/min^{1/2} (RSD = 5.3%)
Average Regression = 0.9777

The following table shows the results of IVRT under the above conditions on Acitretin 0.5% w/w Gel (Example 8).

### Example 8

### Acitretin 0.5% w/w Gel

| | Cell 1 | Cell 2 | Cell 3 | Cell 4 | Cell 5 | Cell 6 |
|---|---|---|---|---|---|---|
| Total Release, | 22.2 | 19.4 | 19.8 | 20.2 | 19.2 | 18.7 |
| % | | | | | | |
| Release Rate | 0.014 | 0.013 | 0.012 | 0.013 | 0.012 | 0.012 |
| (mg/cm² | | | | | | |
| per min^{1/2}) | | | | | | |
| Regression (r) | 0.9830 | 0.9820 | 0.9815 | 0.9806 | 0.9832 | 0.9793 |

Average total release = 19.9%
Average release rate = 0.013 mg/cm²/min^{1/2} (RSD = 6.4%)
Average Regression = 0.9731

The following table shows the results of IVRT under the above conditions on Acitretin 0.5% w/w Gel (Example 9).

### Example 9

### Acitretin 0.5% w/w Gel

| | Cell 1 | Cell 2 | Cell 3 | Cell 4 | Cell 5 | Cell 6 |
|---|---|---|---|---|---|---|
| Total Release, | 23.9 | 20.8 | 24.1 | 22.2 | 20.9 | 21.0 |
| % | | | | | | |
| Release Rate | 0.015 | 0.014 | 0.014 | 0.015 | 0.013 | 0.014 |
| (mg/cm² | | | | | | |
| per min^{1/2}) | | | | | | |
| Regression (r) | 0.9825 | 0.9816 | 0.9832 | 0.9810 | 0.9803 | 0.9771 |

Average total release = 22.1%
Average release rate = 0.014 (RSD = 5.3%)
Average Regression = 0.9810

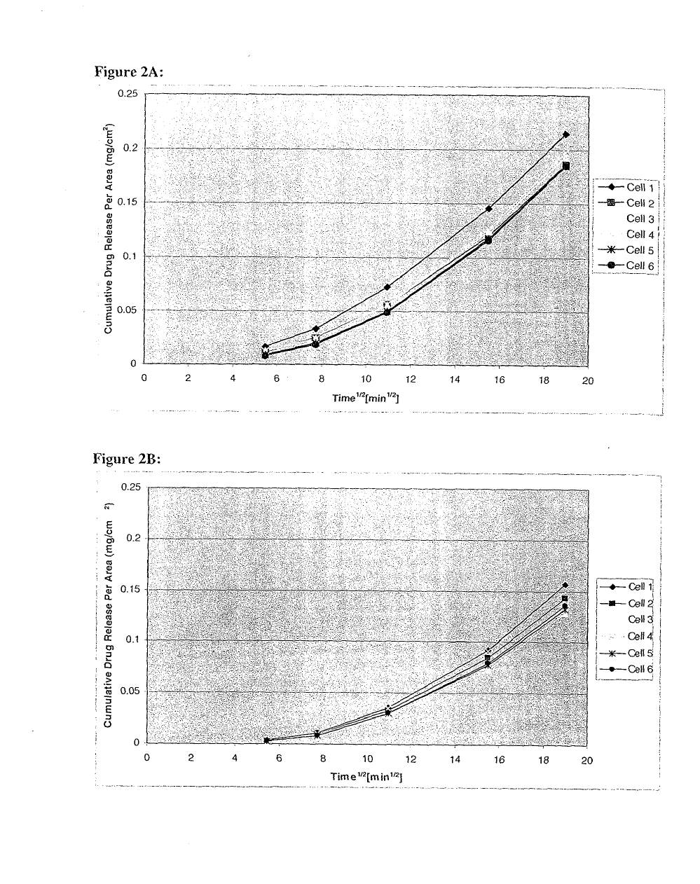

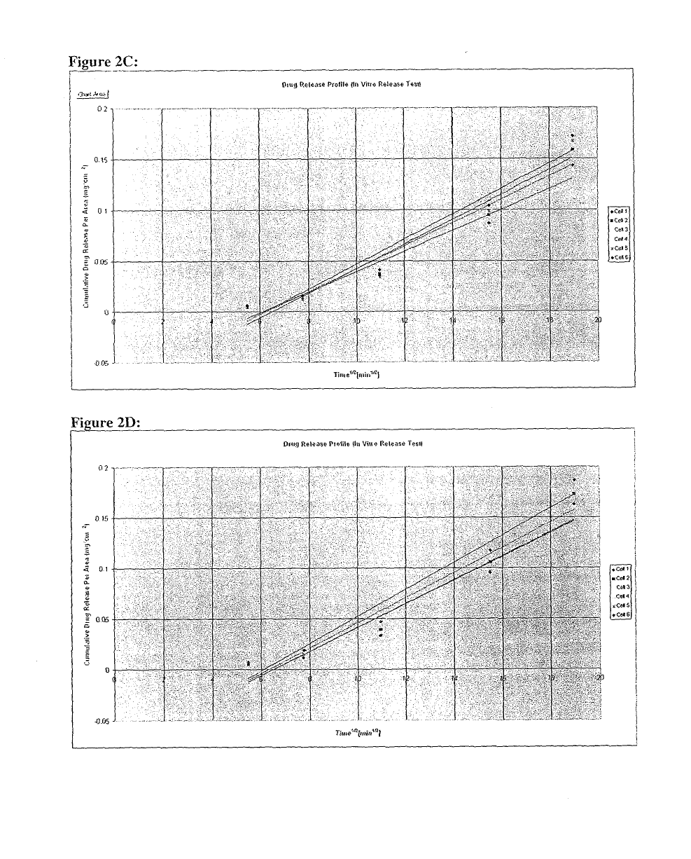

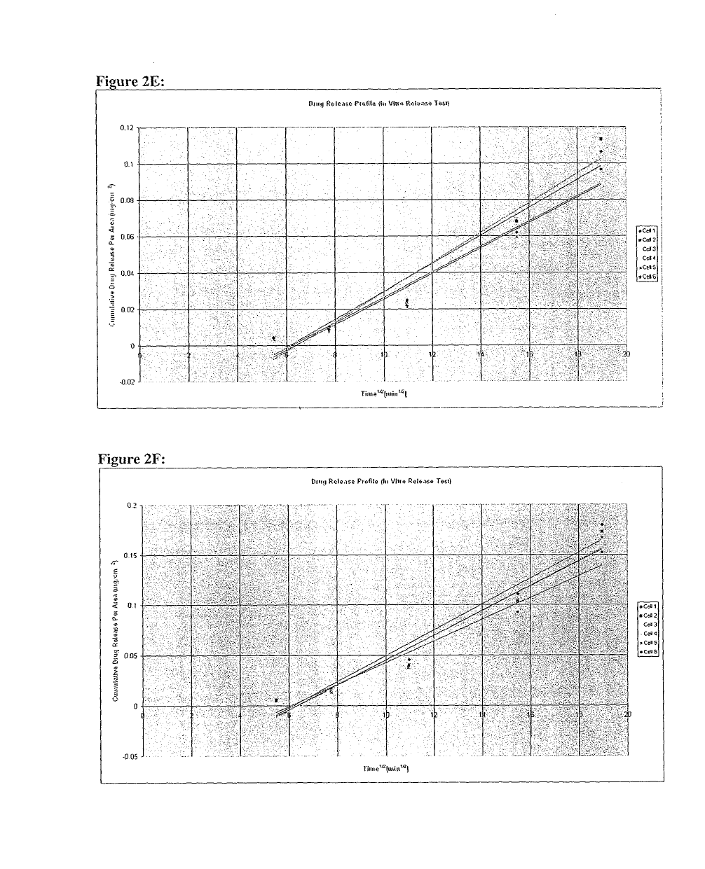

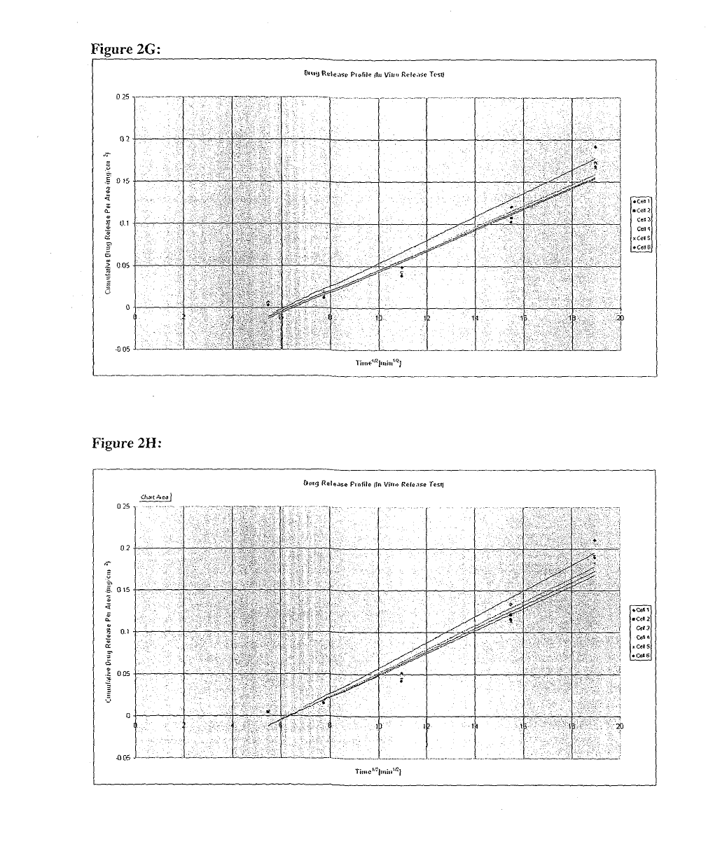

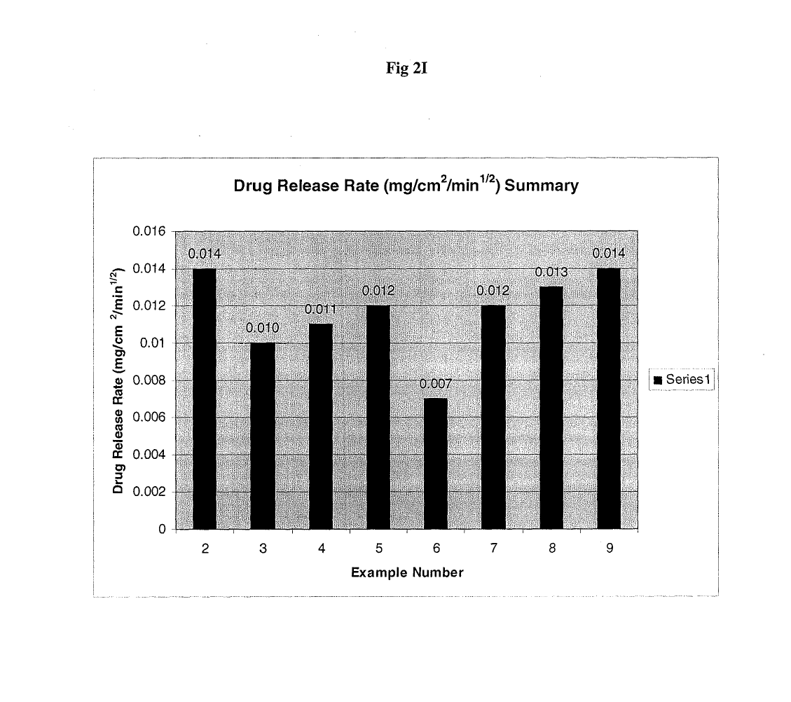

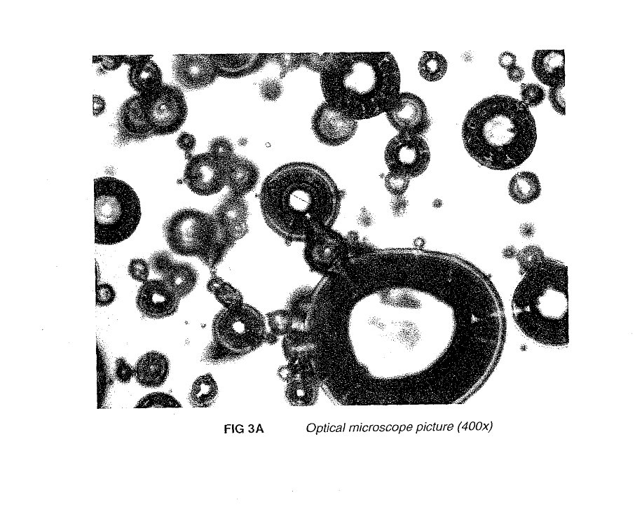

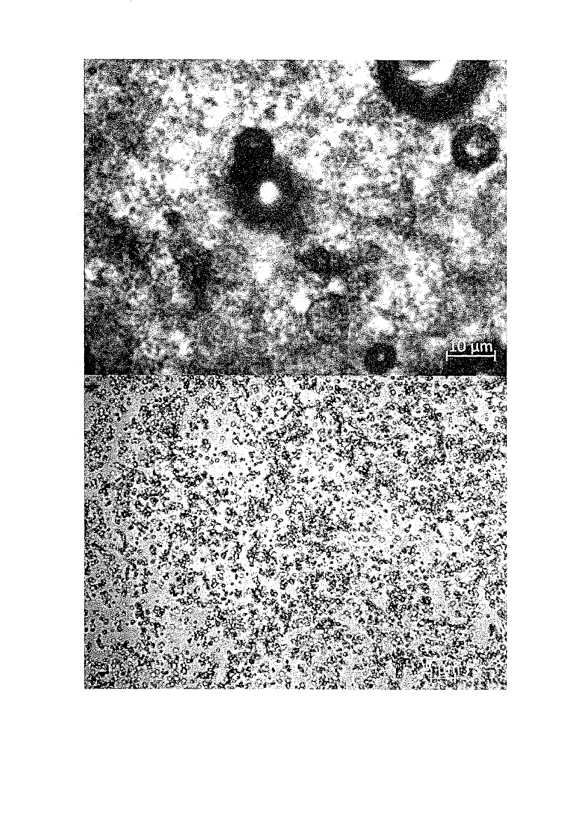

These results are also illustrated in FIGS. 2A to 2H and summarized in FIG. 2I. As can be seen, these formulations achieve an average release rate of not less than 0.01 mg/cm² per min^{1/2} under these conditions, with the exception of Example 6, where a lower release rate has been achieved with a composition containing 0.25% w/w acitretin and 0.6% w/w carbomer. For some purposes, it may be desired to achieve a lower rate of release of acitretin, in association with particular selected properties of for example, viscosity.

Various modifications to and departures from the disclosed example embodiments will occur to those having ordinary skill in the art. The subject matter that is intended to be within the spirit of this invention is set forth in the claims.

1. A topical medicament for reducing at least one symptom of at least one dermatological condition comprising acitretin as a nanosuspension.

2. A topical medicament for reducing at least one symptom of at least one dermatological condition comprising not less than 0.25% w/w acitretin, wherein the medicament shows a release rate of not less than 0.01 mg/cm² per min^{1/2} as measured using a Franz diffusion cell in vitro release testing system utilizing the following conditions: receptor medium comprising 1% DMSO in (35% ethanol: 65% phosphate buffer pH 8.0), speed 700 rpm, membrane polysulfone 0.45 μm, dosage 300±30 mg, temperature 32.5±0.5° C.

3. The medicament of claim 2, comprising a stable nanosuspension of acitretin.

4. The medicament of claim 3, which is a gel.

5. The medicament of claim 3 in which the acitretin is substantially amorphous.

6. The medicament of claim 3 in which at least 90% of the volume distribution of acitretin particles according to the laser diffraction technique are 1 micron or less in diameter.

7. The medicament of claim 3 in which at least 98% of the acitretin particles are 1 micron or less in diameter.

8. The medicament of claim 3 in which at least 99% of the acitretin particles are 1 micron or less in diameter.

9. The medicament of claim 3 comprising a copolymer of vinylpyrrolidone and vinyl acetate.

10. The medicament of claim 3 comprising a spray dried powder comprising a solid dispersion of acitretin in a copolymer of vinylpyrrolidone and vinyl acetate.

11. The medicament of claim 10 wherein the spray dried powder contains from 3% to 25% w/w acitretin.

12. The medicament of claim 10 wherein the w/w % ratio of acitretin to copolymer in the spray dried powder is 5:95 or 25:75, or 20:80, or 15:85, or 12.5:87.5, or 10:90, or 7.5:92.5, or 3:97.

13. The medicament of claim 4 comprising a gelling agent.

14. The medicament of claim 4 comprising a dispersing agent.

15. The medicament of claim 14 where said dispersing agent is a polysorbate.

16. The medicament of claim 14 where the dispersing agent is present at a level of not more than about 0.3% w/w of the medicament.

17. A method of manufacture of the medicament of claim 4 comprising forming a solid dispersion of acitretin and a copolymer of vinylpyrrolidone and vinyl acetate and combining the solid dispersion with an aqueous gel base.

18-19. (canceled)

20. The use of a medicament according to claim 1 for treatment of actinic keratosis.

21. A topical medicament for reducing at least one symptom of at least one dermatological condition comprising at least about 0.5% w/w acitretin, wherein the medicament shows a release rate of not less than 0.01 mg/cm² per min^{1/2} as measured using a Franz diffusion cell in vitro release testing system utilizing the following conditions: receptor medium comprising 1% DMSO in (35% ethanol: 65% phosphate buffer pH 8.0), speed 700 rpm, membrane polysulfone 0.45 μm, dosage 300±30 mg, temperature 32.5±0.5° C.

22. A topical medicament for reducing at least one symptom of at least one dermatological condition comprising acitretin particles as a nanosuspension, wherein at least 90%, by volume, of the acitretin particles suspended are 1 micron or less in size.

23. The topical medicament of claim 22, wherein at least 98%, by volume, of the acitretin particles suspended are 1 micron or less in size.

24. The topical medicament of claim 22, wherein at least 99%, by volume, of the acitretin particles suspended are 1 micron or less in size.

25. The topical medicament of claim 22, wherein the topical medicament is in gel form.

26. The topical medicament of claim 22, wherein the acitretin is a solid dispersion of acitretin with a copolymer.

27. The topical medicament of claim 22, wherein acitretin is present at about 0.25-0.5 w/w.

28. The topical medicament of claim 22, wherein the copolymer is copovidone.

29. The topical medicament of claim 22, further comprising a dispersing agent.

30. The topical medicament of claim 29, wherein the dispersing agent is a polysorbate.

31. The topical medicament of claim 30, wherein the dispersing agent is polysorbate 20 present in an amount of less than about 0.3% w/w.

32. The topical medicament of claim 22, further comprising a chelating agent.

33. The topical medicament of claim 32, wherein the chelating agent is EDTA.

34. The topical medicament of claim 32, wherein the composition comprises less than about 0.3% w/w polysorbate 20, and no EDTA.

35. The topical medicament of claim 32, further comprising EDTA in the absence of polysorbate 20.

36. The topical medicament of claim 32, further comprising EDTA in the presence of less than about 0.1% w/w polysorbate 20.

37. The topical medicament of claim 22, further comprising residual solvent.

38. The topical medicament of claim 37, wherein the residual solvent is THF, and is present in a concentration of at least about 0.4% w/w.

39. The topical medicament of claim 22, further comprising at least one preservative.

40. The topical medicament of claim 39, wherein the preservative is selected from the group consisting of a sodium paraben, sodium methylparaben, sodium propylparaben, potassium sorbate, phenoxyethanol, and combinations thereof.

41. The topical medicament of claim 22, further comprising propylene glycol of about 2.5% to about 5% w/w.

42. The topical medicament of claim 22, wherein the composition comprises carbomer.

43. The topical medicament of claim 22, wherein acitretin is present at about 0.25-0.5 w/w, and the carbomer is between 0.4% and 0.6%.

44. The topical medicament of claim 22, wherein the medicament shows a release rate of not less than 0.01 mg/cm² per min^{1/2} as measured using a Franz diffusion cell in vitro release testing system utilizing the following conditions: receptor medium comprising 1% DMSO in (35% ethanol: 65% phosphate buffer pH 8.0), speed 700 rpm, membrane polysulfone 0.45 μm, dosage 300±30 mg, temperature 32.5±0.5° C.

45. A method of manufacture of the topical medicament of claim 22 which comprises forming a solid dispersion of acitretin particles and a copolymer of vinylpyrrolidone and vinyl acetate by spray drying pre-dissolved acitretin with a copolymer, and combining the solid dispersion with an aqueous gel base.

46-66. (canceled)
